(19) United States
(12) Patent Application Publication
(10) Pub. No.: US 2014/0249524 A1
(43) Pub. Date: Sep. 4, 2014

(54) SYSTEM AND METHOD FOR PERFORMING RENAL NERVE MODULATION

(71) Applicant: BOSTON SCIENTIFIC SCIMED, INC., MAPLE GROVE, MN (US)

(72) Inventor: GORDON J. KOCUR, LINO LAKES, MN (US)

(73) Assignee: BOSTON SCIENTIFIC SCIMED, INC., MAPLE GROVE, MN (US)

(21) Appl. No.: 14/194,491

(22) Filed: Feb. 28, 2014

Related U.S. Application Data
(60) Provisional application No. 61/771,700, filed on Mar. 1, 2013.

(51) Int. Cl. A61B 18/12; A61F 7/12

(52) U.S. Cl. CPC A61B 18/1206; A61F 7/12
USPC 606/40; 606/34; 607/113

(57) ABSTRACT

Systems and methods for monitoring and performing tissue modulation are disclosed. An example system may include an elongate shaft having a distal end region and a proximal end and having at least one ablation electrode disposed adjacent to the distal end region. The system may further include a control unit for controlling the power level and duration of the modulation procedure.

### CROSS-REFERENCE TO RELATED APPLICATIONS

This application claims priority under 35 U.S.C. §119 to U.S. Provisional Application Ser. No. 61/771,700, filed Mar. 1, 2013, the entirety of which is incorporated herein by reference.

### TECHNICAL FIELD

The present disclosure pertains to medical devices and methods for making and using medical devices. More particularly, the present disclosure pertains to medical devices and methods for performing renal nerve modulation.

### BACKGROUND

Certain treatments require the temporary or permanent interruption or modification of select nerve function. One example treatment is renal nerve ablation, which is sometimes used to treat conditions related to congestive heart failure or hypertension. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves, including renal nerves, run along the walls of or in close proximity to blood vessels and thus can be accessed via the blood vessels. In some instances, it may be desirable to ablate perivascular renal nerves using radiofrequency energy. The target nerves must be heated sufficiently to make them nonfunctional; however tissue adjacent to the nerves may also be damaged. It may be desirable to provide for alternative systems and methods for intravascular nerve modulation that reduce damage to surrounding tissues.

### SUMMARY

The disclosure is directed to several alternative designs, materials and methods of manufacturing medical device structures and assemblies for performing nerve ablation and method for performing nerve ablation.

Accordingly, one illustrative embodiment is a method for performing nerve modulation. A nerve modulation system including an electrode adjacent to a distal end of the nerve modulation system and a control unit may be provided. The nerve modulation system may be advanced through a lumen such that the electrode is adjacent to a target region. The electrode may be brought into contact with a wall of the lumen and power then applied to the electrode. A temperature adjacent to the electrode may be measured. The control unit may include a control algorithm for controlling a power level and a duration power is applied to the electrode.

Another illustrative embodiment is a nerve modulation system including an elongate shaft having a proximal end and a distal end. An electrode may be positioned adjacent to the distal end of the elongate shaft. The nerve modulation system may further include a control unit positioned adjacent to the proximal end of the elongate shaft. The control unit may include a control algorithm configured to control power applied to the electrode.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a schematic view illustrating a renal nerve modulation system in situ.

FIG. 2 illustrates a portion of an example renal nerve modulation system.

FIG. 3 illustrates an alternative configuration of the example renal nerve modulation system shown in FIG. 2.

FIG. 4 is a graph of power supplied to the nerve modulation system versus time.

FIG. 5 is a graph of the depth of the 50° C. isotherm versus time.

FIG. 6 is a bar graph illustrating the maximum lesion depth versus tip force and time.

FIG. 7 is a bar graph illustrating the maximum lesion depth for different control parameters.

FIG. 8 is a bar graph illustrating mean renal norepinephrine levels for different control parameters.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

For purposes of this disclosure, “proximal” refers to the end closer to the device operator during use, and “distal” refers to the end farther from the device operator during use.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

Certain treatments require the temporary or permanent interruption or modification of select nerve function. One example treatment is renal nerve ablation, which is sometimes used to treat conditions related to congestive heart failure or hypertension. The kidneys produce a sympathetic response to congestive heart failure, which, among other effects, may increase the undesired retention of water and/or sodium. Ablating some of the nerves running to the kidneys may reduce or eliminate this sympathetic function, which may provide a corresponding reduction in the associated undesired symptoms.

While the devices and methods described herein are discussed relative to renal nerve modulation, it is contemplated that the devices and methods may be used in other locations and/or applications where nerve modulation and/or other tissue modulation including, but not limited to, heating, activation, blocking, disrupting, or ablation are desired, such as, but not limited to: blood vessels, urinary vessels, or in other tissues via trocar and cannula access. For example, the devices and methods described herein can be applied to hyperplastic tissue ablation, tumor ablation, benign prostatic hyperplasia therapy, nerve excitation or blocking or ablation, modulation of muscle activity, hyperthermia or other warming of tissues, etc. In some instances, it may be desirable to ablate perivascular renal nerves with radiofrequency (RF) energy. The term modulation refers to ablation and other techniques that may alter the function of nerves and other tissue such as brain tissue or cardiac tissue. When multiple ablations are desirable, they may be performed sequentially by a single ablation device.

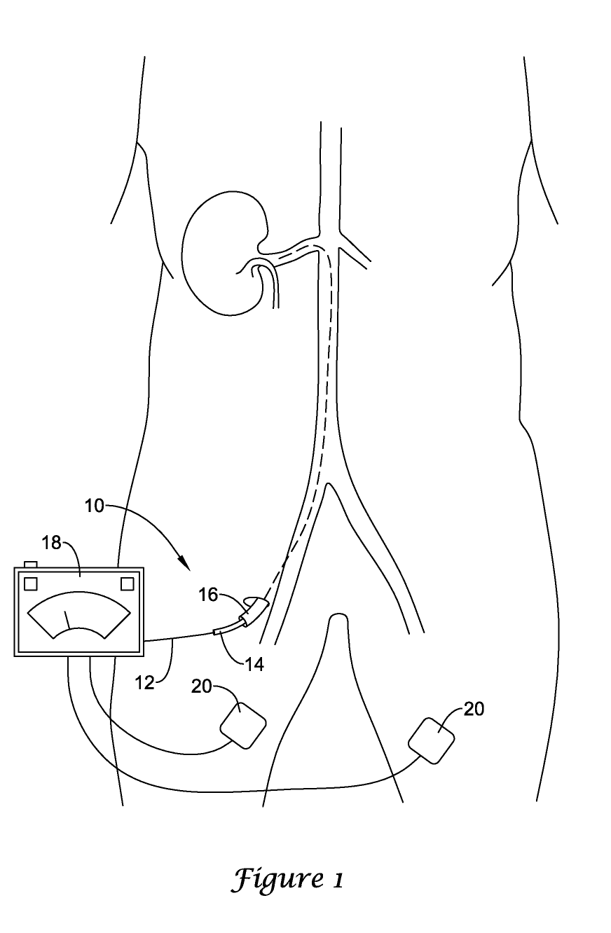

FIG. 1 is a schematic view of an illustrative renal nerve modulation system 10 in situ. System 10 may include one or more conductive power elements 12, a central elongate shaft 14, a guide catheter 16, a control and power block 18 and return electrode patches 20. The control and power element 12 provides power to an electrode or other modulation element disposed adjacent to, about, on, and/or within the central elongate shaft 14 and, optionally, within the guide catheter 16, the details of which can be better seen in subsequent figures. A proximal end of element 12 may be connected to a control and power block 18, which supplies the necessary electrical energy to activate the one or more electrodes at or near a distal end of the element 12. The control and power block 18 may include monitoring elements to monitor parameters such as power, temperature, voltage, pulse size and/or frequency and other suitable parameters as well as suitable controls for performing the desired procedure. In some instances, the power block 18 may control an ablation electrode. The ablation electrode may be configured to operate at a frequency of about 460 kilohertz (KHz). It is contemplated that any desired frequency may be used, for example, from 450-500 KHz. In addition, it is contemplated that frequencies outside this range may also be used, as desired. It is further contemplated that the perivascular nerves may be ablated by other means including application of thermal, ultrasound, laser, microwave, and other related energy sources to the target region and these devices may require that power be supplied by the power block 18 in a different form. In some instances, return electrode patches 20 may be connected to the control and power block 18 and may be supplied on the legs and/or at another conventional location on the patient's body to complete the circuit.

Mammalian anatomy can vary greatly from one specimen to another. Internal studies of human renal anatomy have shown that most nerves (approximately 90%) tend to be within 2 millimeters (mm) of the inner surface of the artery wall. Therefore, it may be desirable to perform renal nerve modulation to a minimum depth of 2 mm. However, as some nerves may be further than 2 mm from the artery wall, it may be desirable to perform nerve modulation to a depth deeper than 2 mm. As the depth of the desired treatment region is expanded, the risk of damaging adjacent structures, such as, but not limited to, the bowel or psoas muscle, increases. Damage to tissue outside of the desired treatment region may cause additional pain for the patient or other medical issues depending on the severity of the damage. Therefore, it may be desirable to limit the depth of the treatment region. For example, it may be desirable to limit the depth of the treatment region to approximately less than 4 mm from the vessel wall.

In order to alter the function of the nerves or other tissue, it may be desirable to heat the target region to a point where the tissue begins to denature or irreversibly change, for example, to approximately 50-65° C. It may be desirable to maintain the temperature of the tissue surrounding the modulation system less than 90° C. (e.g., less than 65° C.) so as to minimize any undesired impact on these tissues. Controlling the electrode temperature below 65° C. may reduce the coagulation of blood on the electrode and artery surface and prevent the blood and/or artery surface from turning brown or black. At temperatures greater than 100° C., the water in the tissue may begin to boil creating steam and causing injury. It is contemplated that the target region may be heated to a minimum of 50° C., the temperature at which the function of the nerves may be altered. It is contemplated that heating the target region (a depth of 4 mm) to the desired temperature range (50-65° C.) in as short of a time as possible may limit the spread of heat beyond the target region due to conduction which can cause undesirable injury to the adjacent structures as well as prevent steam formation.

A variety of different ablation devices may be used to modulate and/or ablate nerve tissue adjacent to a renal artery. While these devices may be configured to ablate tissue across a broad range of depths within a given treatment region and/or raise the temperature of the target tissue any number of degrees, these devices are generally not specifically designed to target nerve tissue while simultaneously minimizing damage to surrounding tissue. For example, a number of ablation devices may be configured to ablate tissue at locations relatively near to a renal artery vessel wall, at locations relatively far from the renal artery vessel wall, and locations in between. However, these devices are not specifically tuned to a particular target depth so that only targeted renal nerves are ablated. Thus, some devices designed to treat across a broad range of tissue depths and/or designed to simply raise the temperature any number of degrees could undesirably effect (e.g., cause damage to) surrounding tissue.

Furthermore, part of targeting nerves at a particular depth or distance away from the vessel wall may include raising the temperature to a particular temperature range at the target location/depth while minimizing the temperature changes at surrounding areas. Therefore, not only is a specific target depth a potentially important factor to consider when performing an intervention (e.g., a renal nerve ablation procedure), the ability to raise the temperature at that depth while reducing temperature changes at surrounding areas may be desirable.

Disclosed herein are devices/systems and method that are designed to aid in a variety of medical interventions including renal nerve ablation procedures. The example devices disclosed herein may be configured to target tissue (e.g., renal nerves) located within a particular range of depths or distances away from the vessel wall of a renal artery. In addition, the example devices may be configured to increase the temperature at the target tissue (e.g., adjacent to the renal nerves) so that potential damage to surrounding tissue can be reduced. A number of structural features of these devices including, for example, deflectability and/or controlled contact force with the vessel, size of the ablation electrode, and the like may also contribute to the ability of these devices to achieve the desired end result. Furthermore, the example methods disclosed herein may be designed to achieve ablation at the desired depth while achieving the desired temperature change. This may include the use of a number of methods, protocols, and/or algorithms that precisely control the power delivered to the ablation device (e.g., ablation electrode), the depth to which ablation occurs, the temperature changes of target and surrounding tissue, the contact force of the ablation member/electrode with the vessel wall, and other factors so as to achieve controlled ablation. Some details regarding these devices and methods are disclosed in more detail herein.

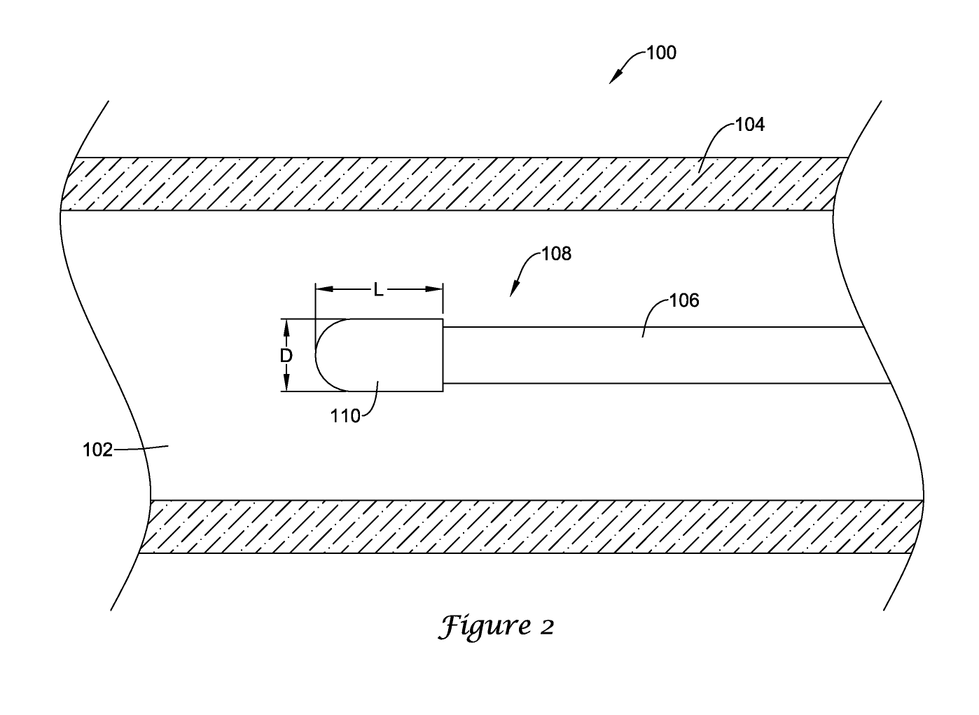

FIG. 2 is an illustrative embodiment of a distal end of a renal nerve modulation system 100 that may be utilized ablate or otherwise module renal nerves while minimizing damage to surrounding tissue. As shown, the renal nerve modulation system 100 may be disposed within a body lumen or vessel 102 having a wall 104. The outer surface of the vessel wall 104 may be surrounded by local body tissue. The local body tissue may comprise adventitia and connective tissues, nerves, fat, fluid, etc. in addition to the muscular vessel wall 104. In some instances, the local body tissue may include bowel and/or psoas muscle tissue. The system 100 may include an elongate shaft 106 having a distal end region 108. The elongate shaft 106 may extend proximally from the distal end region 108 to a proximal end configured to remain outside of a patient's body. The proximal end of the elongate shaft 106 may include a hub attached thereto for connecting other treatment devices or providing a port for facilitating other treatments. It is contemplated that the stiffness of the elongate shaft 106 may be modified to form a modulation system 100 for use in various vessel diameters and various locations within the vascular tree. In some instances, the proximal portion of the elongate shaft 106 may be flexible to enable consistent torque transmission. The elongate shaft 106 may further include one or more lumens extending therethrough. For example, the elongate shaft 106 may include a guidewire lumen and/or one or more auxiliary lumens. The lumens may have a variety of configurations and/or arrangements. For example, the guidewire lumen may extend the entire length of the elongate shaft 106 such as in an over-the-wire catheter or may extend only along a distal portion of the elongate shaft 106 such as in a single operator exchange (SOE) catheter. These examples are not intended to be limiting, but rather examples of some possible configurations. While not explicitly shown, the modulation system 100 may further include temperature sensors/wire, an infusion lumen, radiopaque marker bands, fixed guidewire tip, a guidewire lumen, external sheath and/or other components to facilitate the use and advancement of the system 100 within the vasculature. It is further contemplated that the modulation system 100 may include one or more centering baskets, expandable framework, and/or expandable balloons to center or otherwise position the modulation system 100 within the body lumen 102. In some embodiments, the elongate shaft 106 may include push and/or pull wires to deflect a distal end region 108 of the elongate shaft 106. For example, a push and/or pull wire may be attached adjacent to the distal end of the elongate shaft 106 and then extend along an outer surface of the elongate shaft 106 or along an interior passageway formed in the shaft 106 to a position where it is accessible to a user. In other embodiments, the elongate shaft 106 may incorporate a planar deflection mechanism, such as a rib and spine mechanism. However, it is contemplated that the elongate shaft 106 may be deflected in any desired manner.

The system 100 may include a distal ablation electrode 110 positioned adjacent the distal end region 108 of the elongate shaft. While the ablation electrode 110 is described as a radiofrequency electrode, it is contemplated that other methods and devices for raising the temperature of the nerves may be used, such as, but not limited to: ultrasound, microwave, or other acoustic, optical, electrical current, direct contact heating, or other heating. While the system 100 is illustrated as including one ablation electrode 110, it is contemplated that the modulation system 100 may include any number of ablation electrodes 110 desired, such as, but not limited to, two, three, four, or more. If multiple ablation electrodes 110 are provided, the ablation electrodes 110 may be longitudinally and/or radially and/or circumferentially spaced as desired. In some embodiments, the ablation electrode 110 may include a cylindrical electrode with a hemispherical end adjacent the distal end of the elongate shaft 106. In other instances, the electrode 110 may include wire wrapped coils, generally solid shapes, ball-type electrodes, etc. In some embodiments, the ablation electrode 110 may be formed of a separate structure and attached to the elongate shaft 106. For example, the ablation electrode 110 may be machined or stamped from a monolithic piece of material and subsequently bonded or otherwise attached to the elongate shaft 106. In other embodiments, the ablation electrode 110 may be formed directly on the surface of the elongate shaft 106. For example, the ablation electrode 110 may be plated, printed, or otherwise deposited on the surface. It is contemplated that the ablation electrode 110 may take any shape desired, such as, but not limited to, square, rectangular, circular, elliptical, etc. In some instances, the ablation electrode 110 may be a radiopaque marker band. The ablation electrode 110 may be formed from any suitable material such as, but not limited to, platinum, gold, stainless steel, cobalt alloys, or other non-oxidizing materials. In some instances, titanium, tantalum, or tungsten may be used.

In some embodiments, the ablation electrode 110 may have rounded edges in order to reduce the effects of sharp edges on current density. The size of the ablation electrode 110 may be chosen to optimize the current density without increasing the profile of the modulation system 100. For example, an ablation electrode 110 that is too small may generate high local current densities resulting in higher tissue temperatures with reduced cooling from the blood. An ablation electrode 110 that is too large may require larger, undesirable vascular access devices or be difficult to accurately manipulate and place the tip at the target tissue. In some embodiments, the electrode may have a length L of approximately 0.167 inches (or approximately 4.25 mm) and a width or diameter D of approximately 0.050 inches (or approximately 1.27 mm). It is contemplated that the width of the electrode may be selected to be compatible with a 6 French guide catheter. These are just examples. In some instances, only a portion of the electrode length may be exposed during the ablation procedure. For example, less than 90%, less than 75%, less than 50%, or less than 25% of the total electrode length may be exposed. These ranges are merely exemplary. It is contemplated that any desired length of the electrode may be exposed. In some instances, a longer electrode may further reduce the maximum electrode and tissue temperature for improved safety from blood coagulation, vessel lumen tissue burn, thrombus, and tissue steam pops. It is further contemplated that a longer electrode may run cooler than a shorter electrode due to increased contact area between the electrode and the blood and a more disuse electrical field. In some instances, the ablation electrode 110 may have an aspect ratio of 1.8:1 (length to width) or greater although this is not required. It is completed that the aspect ratio of the ablation electrode 110 may be in the rage of 1:1 to 2.5:1 (length to width) Such an elongated structure may provide the ablation electrode 110 with more surface area without increasing the profile of the modulation system 100. It is contemplated that the ablation electrode 110 may also be sized according to the desired treatment region. For example, in renal applications, the ablation electrode 110 may be sized to be compatible with a 6 French guide catheter, although this is not required.

While not explicitly shown, the ablation electrode 110 may also include other structures and/or features associated typically associated with ablation (e.g., thermal ablation) such as a temperature monitoring member, which may take the form of a thermocouple or thermistor. In at least some embodiments, a thermistor including two thermistor wires may be disposed adjacent to ablation electrode 110. In some embodiments, the wires are not physically connected to ablation electrode 110. The thermistor wires may terminate in a center bore of the ablation electrode 110 and may be potted with a thermally conducting epoxy in a plastic tube which is then glued to the bore of the ablation electrode 110.

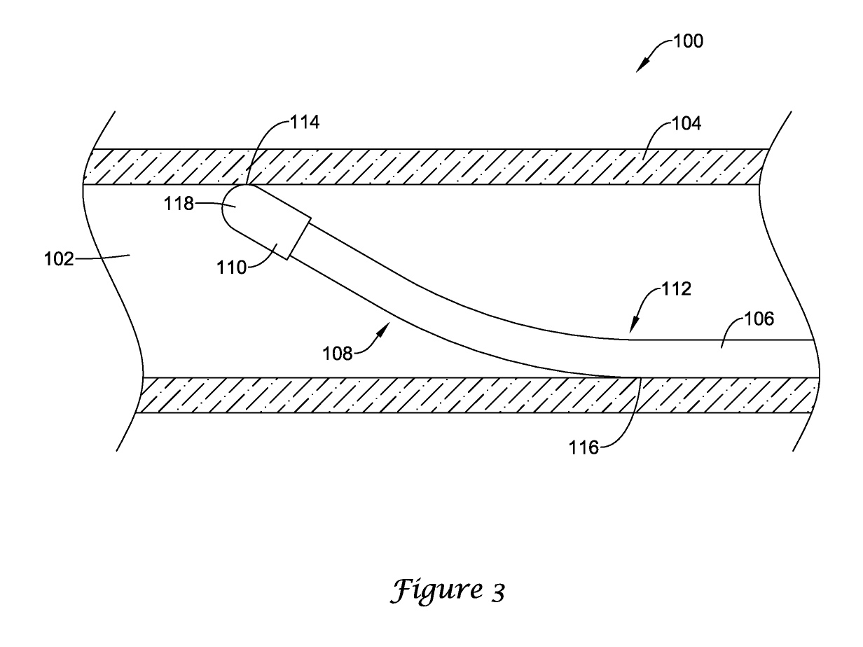

The modulation system 100 may be advanced through the vasculature in any manner known in the art. For example, system 100 may include a guidewire lumen to allow the system 100 to be advanced over a previously located guidewire. In some embodiments, the modulation system 100 may be advanced, or partially advanced, within a guide catheter such as the guide catheter 16 shown in FIG. 1. Once the ablation electrode 110 of the modulation system 100 has been placed adjacent to the desired treatment area, positioning mechanisms may be deployed, if so provided. For example, as shown in FIG. 3, the distal end region 108 may be deflected such that the electrode 110 or electrode tip 118 contacts the vessel wall 104. As discussed above, in some embodiments, the elongate shaft 106 may include push and/or pull wires to deflect a distal end region 108 of the elongate shaft 106. For example, a push and/or pull wire may be attached adjacent to the distal end of the elongate shaft 106 and then extend along an outer surface of the elongate shaft 106 or along an interior passageway formed in the shaft 106 to a position where it is accessible to a user. In other embodiments, the elongate shaft 106 may incorporate a planar deflection mechanism, such as a rib and spine mechanism. In some instances, the modulation system 100 may be structured such that an operator may have the ability to deflect the elongate shaft 106 such that the force with which the ablation electrode 110 or electrode tip 118 contact the vessel wall 104 may be controlled. For example, it may be desirable for the operator to apply a tip force in the range of 5 to 30 grams (g). This may allow the operator to vary the electrode area contact in varying anatomies, vessel diameters and positions along the vessel. This may ensure a greater number of ablations make adequate, stable electrode contact to deliver repeatable optimal lesions. However, it is contemplated that the elongate shaft 106 may be deflected in any desired manner.

In some instances, multiple treatments may be used to achieve the desired tissue modulation. In some instances, the elongate shaft 106 may be rotated and additional ablation can be performed at multiple locations around the circumference of the vessel 102. The number of times the elongate shaft 106 is rotated at a given longitudinal location may be determined by the number and size of the ablation electrodes 110 on the elongate shaft 106. Once a particular location has been ablated, it may be desirable to perform further ablation procedures at different longitudinal locations. If necessary, the elongate shaft 106 may be rotated to perform ablation around the circumference of the vessel 102 at each longitudinal location. This process may be repeated at any number of longitudinal locations desired. In some instances, the treatment may be performed in a helical pattern such that each treatment region is longitudinally and radially spaced from the adjacent treatment region.

During an ablation procedure when the electrode 110 contacts tissue and energy is supplied to the electrode 110, the tissue begins heat. As ablation continues, the electrode 110 also may begin to heat. If the contact between the vessel wall 104 and the electrode 110 is not sufficient, blood may pass over the surface of the electrode 110 thus cooling the electrode 110. The temperature at and/or adjacent to the electrode 110 may be measured and the measurement may be used to help determine if there is sufficient contact between the electrode 110 and the vessel wall 104. For example, a thermistor may be used to monitor the temperature at the electrode. If the temperature is below a predetermined threshold at a predetermined time point at a predetermined power, an error code or message may be generated by or at the control unit (such as control unit 18, shown in FIG. 1) alerting the user. In some embodiments, if the temperature at or adjacent to the electrode 110 is less than 43° C. at 5 seconds, the control unit 18 may determine the tip is not heating and alert the user. For example, in some instances, the user may receive a message indicating the electrode 110 needs to be repositioned.

It is further contemplated that the tip contact between the electrode 110 and the vessel wall 104 may be monitored using a low power to monitor small increases in the temperature of the electrode 110. For example, before the ablation procedure is performed, power may be supplied to the electrode 110 at a power less than the treatment power. If the temperature at or adjacent to the electrode 110 does not increase at least 1° C. within 5 seconds, the control unit 18 may determine the tip is not heating and alert the user. For example, in some instances, the user may receive a message indicating the electrode needs to be repositioned.

In some embodiments, the distal end region 108 may be deflected such that the electrode tip 118 exerts a force on the inner surface of the vessel wall 104. Exerting a force against the vessel wall 104 may improve the stability of the modulation system 100 during the ablation procedure and the contact between the vessel wall 104 and the electrode 110. For example, positioning the electrode tip 118 against a first side 114 of the vessel wall 104 and an intermediate region 112 of the elongate shaft 106 against a second side 116 of the vessel wall 104 may help prevent the elongate shaft 106 from moving during the procedure. For example, the opposite outward forces of the electrode tip 118 and the intermediate region 112 on the inner surface of the vessel wall 104 may act as a gripping mechanism. It is further contemplated that deflecting the distal end region 108 such that the electrode tip 118 exerts a force on the vessel wall may achieve better and/or more consistent electrode 110 to vessel wall 104 contact. In some instances it may be desirable for the operator to apply a tip force in the range of 5 to 30 grams (g).

The force with which the electrode tip 118 contacts the vessel wall 104 may affect the starting impedance. The impedance of the vessel wall 104 may be greater than the impedance of the blood, or surrounding fluid. For example, the vessel wall 104 and/or surrounding tissue may have an impedance of approximately 300-400 ohms (Ω) while blood may have an impedance in the range of approximately 150-180Ω. It is contemplated that the starting impedance may be approximated based on the percentage of the electrode 110 which contacts the vessel wall 104. For example, the measured impedance may increase with increasing tissue contact. Thus, the greater the contact force (and thus the greater the surface area of the electrode 110 contacting the wall 104) the higher the starting impedance may be as more current travels through the higher impedance tissue from the electrode 110. Therefore, the starting impedance may be used to determine an approximate tip force. It is contemplated that it may be desirable for the starting impedance to be at least 40Ω above the impedance of the blood and at least 100Ω below the impedance of the surrounding tissue.

The amount of change of the impedance may depend on the frequency at which the impedance is measured. For example, when the impedance is measured at a lower frequency, such as, but not limited to, 46 KHz, the change in impedance with increasing tip force may be larger than the change in impedance measured at a higher frequency, such as, but not limited to 460 KHz. Measuring the impedance at a lower frequency may provide a more sensitive reading, thus allowing the operator greater control over the force with which the electrode tip 118 contacts the vessel wall 104.

A low tip force in which the electrode tip 118 has minimal contact with the vessel wall 104 (or other tissue) may have starting impedance similar to the impedance of the blood flowing through the vessel. It is contemplated that the due to the minimal tip contact with the tissue, the maximum ablation temperature of the target region may be low, for example, less than 50° C. A medium tip force, in which, for example, approximately 25% of the electrode tip 118 area contacts the vessel wall 104 (or other tissue), may have a starting impedance in the range of 200-225Ω. However, this range is merely exemplary and may vary based on the impedance of the tissue and/or blood. It is contemplated that the maximum ablation temperature of the target region may be between 50° C. and 60° C. when a medium tip force is utilized. A high tip force, in which, for example, approximately 50% of the electrode tip 118 area contacts the vessel wall 104 (or other tissue), may have a starting impedance in the range of 250-265Ω However, this range is merely exemplary and may vary based on the impedance of the tissue and/or blood. It is contemplated that the maximum ablation temperature of the target region may be limited by a control algorithm when a high tip force is used. For example, a control algorithm may be configured to limit the maximum temperature at or adjacent to the electrode tip 118 to less than 65° C., although this is not required. It is contemplated that the maximum temperature may be selected based on the desired treatment.

It is contemplated that the surface area of the electrode 110 that contacts the vessel wall 104 may impact how quickly or to what degree the surrounding tissue is heated. When the electrode tip 118 does not contact the vessel wall 104 or minimally contacts the wall, a small fraction of the current may flow from the electrode 110 through the vessel wall 104 and to the desired treatment region. Therefore, the desired treatment region may not reach the target temperature required to perform tissue modulation. When a large fraction of the electrode tip 118 contacts the vessel wall 104, a large fraction of the current may pass from the electrode 110 through the vessel wall 104 and to the desired treatment region. In this situation, the temperature of the target region may become too hot or tissues outside of the desired target region may be damaged as the heat penetrates deeper than the desired target region. Therefore, it may be desirable to control the force with which the electrode tip 118 contacts the vessel wall 104, and thus the amount of surface area of the electrode 110 contacting the vessel wall 104. Because there is anatomy variability it may be desirable to give the operator a range of tip force to ensure adequate tip contact while limiting the maximum tip force and tip power to minimize excessive heating and undesirable tissue injury. For example, it is contemplated the tip force with which the electrode tip 118 contact the vessel wall 104 may range from approximately 5 to 30 grams

While not explicitly shown, the ablation electrode 110 may be connected to a control unit or to separate control units (such as control unit 18 in FIG. 1) by electrical conductors. Once the modulation system 100 has been advanced to the treatment region, energy may be supplied to the ablation electrode 110. The amount of energy delivered to the ablation electrode 110 may be determined by the desired treatment as well as the feedback provided by the system 100. As energy is supplied to the electrode 110 the surrounding tissues begin to heat. The longer energy is supplied to the electrode 110, the further the heat penetrates the surrounding tissue. In one example, it is contemplated that the maximum tissue depth from the outer surface of the vessel wall 104 that reaches 50° C. may be approximately 6.3 millimeters after energy has been supplied to the electrode 110 at 8 Watts (W) for 120 seconds. It is contemplated that the length of the treatment time may be determined by how quickly the target region reaches the target temperature of at least 50° C.

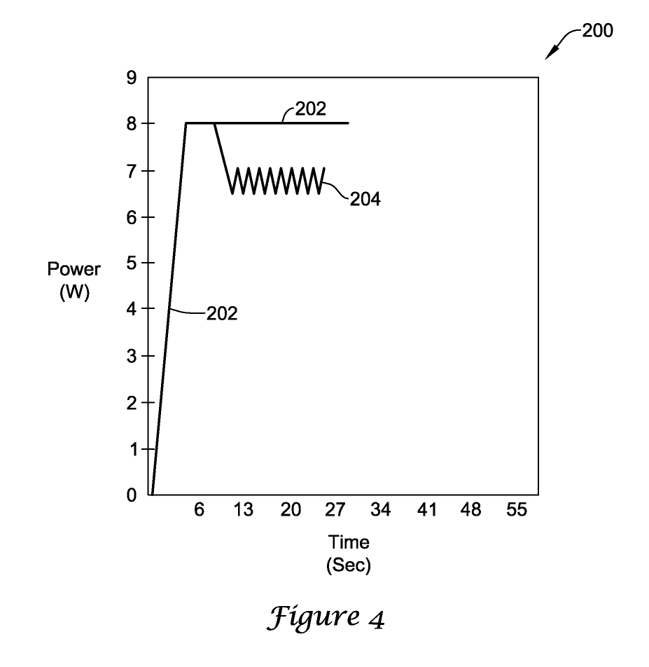

FIG. 4 illustrates a graph 200 of two exemplary control programs that the control and power block may use to control the maximum temperature of the target region and the depth of the resulting lesion. In some instances, the control and power block 18 may be configured and/or programmed to gradually increase the power supplied to the electrode 110. For example, the control and power block 18 may be configured to increase the power from zero to 8 W over a 5 second time frame, as shown by curve 202. In some instances, the power ramp may be linear, although this is not required. In other instances, the power ramp may be exponential. Once a maximum power, for example 8 W, has been reached, the control unit 18 may be configured to maintain a constant power supply 202 for the duration of the treatment. In some instances, the maximum power may be as low as 6 W and as high as 10 W. Increasing the power level may increase the risk of achieving temperatures greater than 90° C. and vessel spasms while decreasing the power level may not cause denervation. The upper limit to the power level may be determined, at least in part, by the electrode 110 size. It is contemplated that given a modulation system, such as modulation system 100, the treatment may be completed in 75 seconds or less, 45 seconds or less, or 30 seconds or less. In some instances, the treatment may be completed in approximately 30 seconds.

In some instances, the control unit 18 may include alternative control algorithms for different operating conditions. For example, if the electrode tip 118 is overheating, for example, if the electrode tip 118 has a temperature greater than 65° C., the control unit 18 may operate using an alternative algorithm 204. In some instances, when a temperature greater than 65° C. is measured, the control unit 18 may decrease the power supplied to the electrode 110 as shown in curve 204 in FIG. 4. It is contemplated that the control unit 18 may fluctuate the power level as necessary to maintain the temperature at less than 65° C. While curve 204 illustrates the power level as fluctuating between approximately 6.5 and 7.0 W, it is contemplated that the control unit 18 may adjust the power level higher or lower as necessary to maintain the desired temperature. It is further contemplated that the power level may be adjusted in any positive or negative increment desired such as, but not limited to 0.1 W, 0.25 W, 0.5 W, 1 W etc.

In some embodiments, the control unit 18 may be further configured to stop ablation if an upper temperature limit is reached. For example, the control unit 18 may be configured to stop ablation if a temperature of 75° C. is reached at the electrode tip 118. In some embodiments, the control unit 18 may be configured to provide a visual or audio alarm, such as an error code or message, to alert the user that the electrode tip 118 is not heating. For example, if the electrode tip 118 has not reach a predetermined temperature, such as, but not limited to, 43° C., by a predetermined time, such as, but not limited to, 5 seconds, the user may be alerted that the tip 118 is not heating and the contact between the electrode tip 118 and the vessel wall 104 is not sufficient. The user may then stop the ablation procedure, if the control unit 18 has not done so automatically, and reposition the electrode tip 118. As noted above, the starting impedance may be measured and used a guideline for determining the approximate tip 118 contact area and/or force with which the electrode tip 118 contacts the vessel wall 104. In some instances, a higher electrode tip 118 to vessel wall 104 contact force may increase the probability of achieving adequate tip contact and energy delivery without generating alarms.

In some instances, the electrode tip 118 may shift during the modulation procedure. The control unit 18 may monitor the temperature at or adjacent to the electrode 110 to determine if the electrode tip 118 has lost contact with the vessel wall 104. For example, if the temperature drops 7° C. or more (or other predetermined amount) for more than 5 seconds (or other predetermined threshold), the ablation procedure may be stopped. A time frame of approximately 10 seconds or time averaging may help reduce faults that may be triggered by brief losses of contact due to respiration, heart beats, or other reasons.

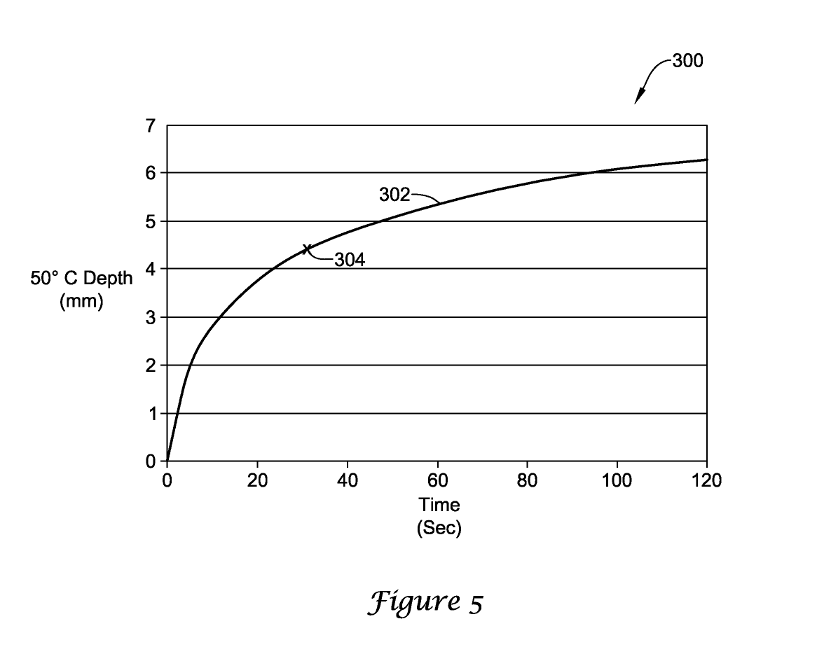

It is contemplated that the duration of the procedure may be determined by the desired procedure. For example, in some instances, the treatment may continue until a desired temperature reaches a desired depth. In some embodiments, the treatment may continue until a desired treatment region reaches 50° C. FIG. 5 illustrates a graph 300 of the depth versus time for 50° C. isotherm. The graph 300 may illustrate the length of time it takes for a modulation system, such as modulation system 100, to heat the tissue surrounding a vessel 102 to 50° C. As can be seen at line 302, the tissue closest to the vessel wall 104 quickly heats to a temperature of 50° C. while tissue further away takes a longer period of time to heat the 50° C. As can be seen, the 50° C. isotherm 302 may reach a depth of just over 4 mm at approximately 30 seconds as shown in point 304. It is noted, that the tissue closest to the vessel wall 104 will be heated to a temperature above 50° C. at a time of 30 seconds. In some instances, if the ablation procedure were to continue beyond 30 seconds it is contemplated that tissue outside the desired target region may rise above 50° C. to 65° C. and be damaged or tissue adjacent to the vessel wall 104 may be heated to the point of boiling.

### EXAMPLES

### Example 1

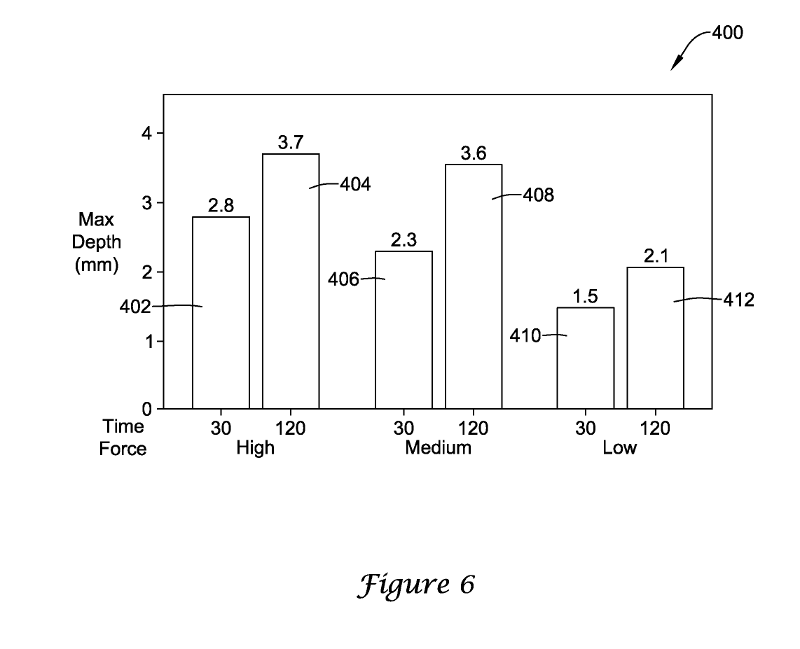

The effect of tip force and length of time of the treatment was studied on bench tissue. Swine myocardial tissue was placed in a 37° C. salt solution. Power was applied to the electrode at 8 Watts with a temperature limit of 70° C. The maximum depth of the lesion was measured for two procedure times, 30 seconds and 120 seconds for a high tip force having an impedance of approximately 260 ohms and approximately greater than 50% area contact, a medium tip force having an impedance of approximately 220 ohms and approximately 25% area contact, and a low tip force having an impedance of approximately 190 ohms. As noted above, the amount of surface area of electrode contacting the vessel wall and/or treatment area is proportional to the tip force. As such, impedance may be used to determine the approximate surface area contacting the treatment region and thus the approximate tip force. FIG. 6 is a bar graph 400 illustrating the results from the experiment. As can be seen at bar 402, a high tip force for a treatment time of 30 seconds yields an average maximum lesion depth of approximately 2.8 mm. A high tip force for a treatment time of 120 seconds yields an average maximum lesion depth of approximately 3.7 mm, as shown at bar 404. A medium tip force for a treatment time of 30 seconds yields an average maximum lesion depth of approximately 2.3 mm, as shown at bar 406. A medium tip force for a treatment time of 120 seconds yields an average maximum lesion depth of approximately 3.6 mm, as shown at bar 408. A low tip force for a treatment time of 30 seconds yields an average maximum lesion depth of approximately 1.5 mm, as shown at bar 410. A low tip force for a treatment time of 120 seconds yields an average maximum lesion depth of approximately 2.1 mm, as shown at bar 412. It is contemplated that a low tip force may not create a lesion deep enough to modulate the desired area and thus may not be as effective as a medium or high tip force. It is further contemplated that the electrode should contact the vessel wall with a force of greater than 5 grams in treatment anatomies.

### Example 2

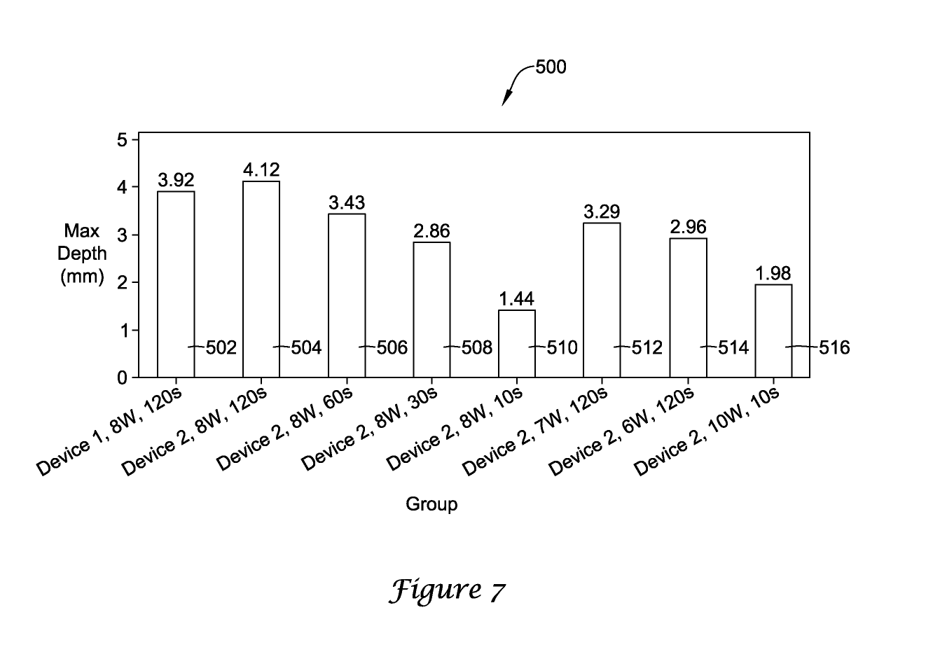

The effect of time and power on lesion depth and lesion volume was studied on two separate devices. The results are summarized in graph 500 (lesion depth) shown in FIGS. 7. Device 1 was a modulation system not embodied by the current disclosure. Device 2 was a device embodied by the current disclosure, such as the device described with respect to FIGS. 2 and 3. The experiment was performed on swine myocardial tissue during a bench test. The myocardial tissue was placed in a 37° C. salt solution. The length of time the energy was applied to the electrode as well as the power level was varied. A starting impedance of 220 ohms was targeted.

In a first test, Device 1 was used to perform tissue modulation on swine myocardial tissue. Energy was supplied to the electrode at 8 W for 120 seconds. As shown at bar 502 in FIG. 7, the average maximum depth was 3.924 mm. In a second test, Device 2 was used to perform tissue modulation on swine myocardial tissue. Energy was supplied to the electrode at 8 W for 120 seconds. As shown at bar 504 in FIG. 7, the average maximum depth was 4.127 mm. In a third test, Device 2 was used to perform tissue modulation on swine myocardial tissue. Energy was supplied to the electrode at 8 W for 60 seconds. As shown at bar 506 in FIG. 7, the average maximum depth was 3.433 mm. In a fourth test, Device 2 was used to perform tissue modulation on swine myocardial tissue. Energy was supplied to the electrode at 8 W for 30 seconds. As shown at bar 508 in FIG. 7, the average maximum depth was 2.86 mm. In a fifth test, Device 2 was used to perform tissue modulation on swine myocardial tissue. Energy was supplied to the electrode at 8 W for 10 seconds. As shown at bar 510 in FIG. 7, the average maximum depth was 1.448 mm. In a sixth test, Device 2 was used to perform tissue modulation on swine myocardial tissue. Energy was supplied to the electrode at 7 W for 120 seconds. As shown at bar 512 in FIG. 7, the average maximum depth was 3.29 mm. In a seventh test, Device 2 was used to perform tissue modulation on swine myocardial tissue. Energy was supplied to the electrode at 6 W for 120 seconds. As shown at bar 514 in FIG. 7, the average maximum depth was 2.965 mm. In an eighth test, Device 2 was used to perform tissue modulation on swine myocardial tissue. Energy was supplied to the electrode at 10 W for 10 seconds. As shown at bar 516 in FIG. 7, the average maximum depth was 1.985 mm. The lesion depths were compared by analysis of variance (ANOVA) with p-value of less than 0.5.

Overall, the experiments illustrate that reducing the time of the procedure or reducing the power decreases the lesion depth and volume. Reducing the tip force can also reduce the lesion depth. As can be seen, the fourth test (Device 2, 8 W, 30 seconds) appears to provide a lesion that is deep enough to ablate to the minimum depth of 2 mm but not so deep as to cause undesirable damage to surrounding tissues. The fourth test also minimizes the duration of the test procedure (as compared to the seventh test) which may help reduce side effects from an extended procedure. For example, a shorter procedure duration may help reduce the duration of pain experienced by patients during treatment, the overall procedure time, errors due to loss of contact and may reduce the maximum temperature of the tissue immediately adjacent to the electrode.

### Example 3

Renal tissue norepinephrine levels may provide an indication of the efficacy of the renal nerve ablation procedure. For example, a decrease in mean kidney norepinephrine may indicate that the function of the nerves has been altered to impact the sympathetic function of the nerves. Swine renal arteries were treated using a helical pattern where treatment regions were spaced by 5 mm and 90 degrees. The number of treatments were determined by the artery length and ranged from four to eight treatments per artery. The animals were treated with device 1 with a maximum power of 8 Watts over 120 seconds. The animals were treated with device 2 with a maximum power of 8 Watts over 30 seconds. Both devices were operated at 460 KHz and a temperature limit of 70° C.

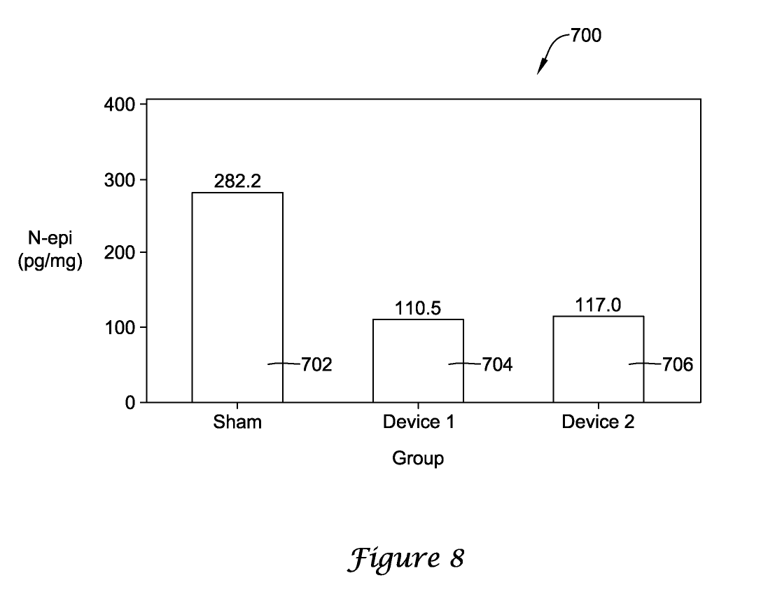

The mean kidney norepinephrine levels were measured 7 days after treatment. The results are summarized in Graph 700 illustrated in FIG. 8. The “Sham” column 702 represents the norepinephrine level of animals that did not receive energy delivery through the catheter. Device 1 was a modulation system not embodied by the current disclosure. Device 2 was a device embodied by the current disclosure, such as the device described with respect to FIGS. 2 and 3.

As can be seen at bar 704, Device 1, operated for 120 seconds at 8 W, had an approximately 61% reduction in norepinephrine relative to the Sham group. Device 2, operated for 30 seconds at 8 W, had an approximately 59% reduction in norepinephrine, as shown at bar 706. The two treatment groups were not significantly different from each other but both were significantly lower than the “Sham” group by students t-test, p<0.5. Treatment times less than 120 seconds may be efficacious, reduce procedure time, patient pain, and injury to structures like the psoas muscle, which can be close to the renal artery.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A method for performing nerve modulation, the method comprising:
providing a nerve modulation system, the nerve modulation system comprising:
an elongate shaft having a proximal end and a distal end;
an electrode positioned adjacent to the distal end of the elongate shaft; and
a control unit positioned adjacent to the proximal end of the elongate shaft;
advancing the nerve modulation system through a lumen such that the electrode is adjacent to a target region;
contacting a wall of the lumen with the electrode;
applying power to the electrode; and
measuring a temperature adjacent to the electrode;
wherein the control unit includes a control algorithm for controlling a power level and a duration power is applied to the electrode.

2. The method of claim 1, wherein the control unit adjusts the power level applied to the electrode to achieve a measured temperature in the range of 50° C. to 65° C. at the target region.

3. The method of claim 1, wherein the power is applied to the electrode in the range of 6-10 Watts.

4. The method of claim 1, wherein the power is applied to the electrode at 8 Watts.

5. The method of claim 1, wherein the electrode exerts a force on the wall of the lumen.

6. The method of claim 5, wherein the force exerted on the wall of the lumen by the electrode is estimated by measuring the impedance of the system prior to applying power to the electrode.

7. The method of claim 1, wherein the control unit applies power to the electrode for 75 seconds or less.

8. The method of claim 1, wherein the control unit ramps the power applied to the electrode from zero Watts to 8 Watts over a predetermined time period.

9. The method of claim 8, wherein the control unit ramps the power applied to the electrode in a linear manner.

10. The method of claim 8, wherein the control unit ramps the power applied to the electrode in an exponential manner.

11. The method of claim 8, wherein once the power applied to the electrode reaches 8 Watts, the control unit maintains the power applied to the electrode at 8 Watts for the duration of the first predetermined time period.

12. The method of claim 1, wherein if the measured temperature exceeds a predetermined maximum threshold, the control unit reduces the power level applied to the electrode.

13. The method of claim 1, wherein if the measured temperature is less than a predetermined minimum threshold at a predetermined time point, the control unit generates an error message.

14. The method of claim 1, wherein if the measured temperature or time averaged temperature drops by more than a predetermined amount for longer than a predetermined time frame, the control unit stops applying power.

15. A method for performing nerve modulation, the method comprising:
providing a nerve modulation system, the nerve modulation system comprising:
an elongate shaft having a proximal end and a distal end;
an electrode positioned adjacent to the distal end of the elongate shaft; and
a control unit positioned adjacent to the proximal end of the elongate shaft;
advancing the nerve modulation system through a lumen such that the electrode is adjacent to a target region, the target region in the range of 2-4 mm from a wall of the lumen;
contacting a wall of the lumen with the electrode such that the electrode exerts a force on the wall of the lumen and a measured starting impedance of the system is at least 40 ohms above an impedance level of blood before wall contact and at least 100 ohms below an impedance of surrounding tissues;
applying power to electrode in a linear manner from 0 Watts to 8 Watts over a time period of 5 seconds;
once the power has ramped from 0 Watts to 8 Watts, applying power to the electrode at 8 Watts for 25 seconds or less to achieve temperature in the range of 50° C. to 65° C. at the target region; and
measuring a temperature adjacent to the electrode;
reducing the power to a level less than 8 Watts if the measured temperature exceeds a predetermined maximum threshold; and
generating a lesion in the target region.

16. A nerve modulation system, the nerve modulation system comprising:
an elongate shaft having a proximal end and a distal end;
an electrode positioned adjacent to the distal end of the elongate shaft; and
a control unit positioned adjacent to the proximal end of the elongate shaft;
wherein the control unit includes a control algorithm configured to control power applied to the electrode.

17. The nerve modulation system of claim 16, further comprising a positioning mechanism configured to deflect the distal end of the elongate shaft away from a longitudinal axis of the elongate shaft.

18. The nerve modulation system of claim 16, wherein the electrode has a length and a width.

19. The nerve modulation system of claim 18, wherein the length of the electrode is greater than the width of the electrode.

20. The nerve modulation system of claim 17, wherein the distal end of the elongate shaft is deflected such that the electrode is positional to exert a force on a wall of a lumen.
